(19) United States
(12) Patent Application Publication
(10) Pub. No.: US 2005/0101970 A1
(43) Pub. Date: May 12, 2005

(54) Functional image-guided placement of bone screws, path optimization and orthopedic surgery

(76) Inventor: William S. Rosenberg, Overland Park, KS (US)

Correspondence Address: Peter L. Berger, Esq.;Levisohn, Berger & Langsam, LLP, 19th Floor, 805 Third Avenue, New York, NY, 10022, US

(21) Appl. No.: 10/982,447

(22) Filed: Nov. 5, 2004

Related U.S. Application Data
(60) Provisional application No. 60/517,688, filed on Nov. 6, 2003.

(51) Int. Cl. A61B 17/58

(52) U.S. Cl. 606/130

(57) ABSTRACT

An intraoperative image-guidance system for real-time visualization of non-anatomic bone properties utilizing hardware, software and display means to determine such properties as bone density or obstructions to surgical instrument movement. Real-time determination of such properties enhances the surgical procedure by ensuring desirable screw placement optimized for pullout strength or using SPECT to determine minimally-invasive surgical paths.

### RELATED APPLICATIONS

This is a nonprovisional application based upon provisional application Ser. No. 60/517,688, filed Oct. 6, 2003, the contents of which are incorporated herein.

### BACKGROUND

This invention relates to a combination of hardware and software allowing a surgeon to utilize intraoperative image-guidance for real-time visualization of non-anatomic bone properties, such as bone density, allowing functional screw path optimization or for permitting preferred instrument path movement for fine surgery.

This invention can be used for surgery anywhere in the body in which skeletal image-guidance would be useful.

Image-guided surgery is the application of radiological imaging to the real-time needs of surgery. In modern usage, this is usually the use of a computer workstation and some method of tracking patient anatomy and surgical instruments to display anatomic positioning. This is often done in multiple planes or using three dimensional rendering.

Image-guided surgery can be useful in the placement of bone screws. This is the case when limited anatomy is available for reference and orientation (such as during minimally-invasive techniques). In addition, image-guidance can minimize risk of injury to important structures in close proximity to the screw path (such as during C1/2 transarticular fixation).

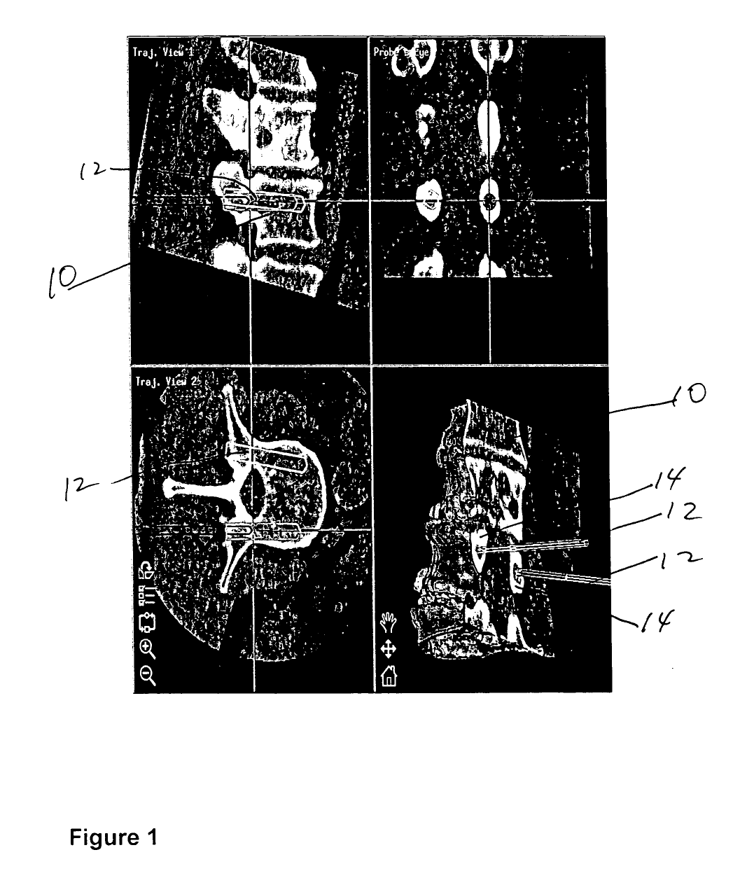

FIG. 1 is a screen shot from StealthStation (Medtronic SNT), during placement of pedicle screws in the spine. The photographs in FIG. 1 show the spine 10, as well as imaging of pedicle screws 12, as well as a rear view of the spine (lower right quadrant) showing imaging of screws 12 into bone structure 14. While this type of image-guided surgery is helpful, it has material limitations in terms of advanced surgical techniques for fine surgery.

To date, image-guided orthopedic surgery has focused on delivering information regarding anatomic details. While sometimes useful (see above) in placing devices safely, clinical results usually depend upon optimizing device pullout strength. In the general case of a bone screw, this is dependent on bone density and screw length. While bone density information would be useful to the surgeon in choosing an optimal screw path with regard to pullout strength, no current system provides anatomic bone density information real-time in the operating room.

There is only one method of anatomically determining bone density: quantitative computed tomography (QCT). Other methods produce results reflecting global, non-specific skeletal bone density. In QCT, the scan is produced with a series of standards placed under the subject in tubes. For each slice, a regression is calculated, using these standards, and a function is generated allowing Hounsfield units to be converted into actual bone density. This is currently used clinically to calculate the overall bone density for a region-of-interest. This approach will, in accordance with this invention be used to determine bone density for each pixel or voxel.

### DESCRIPTION OF INVENTION

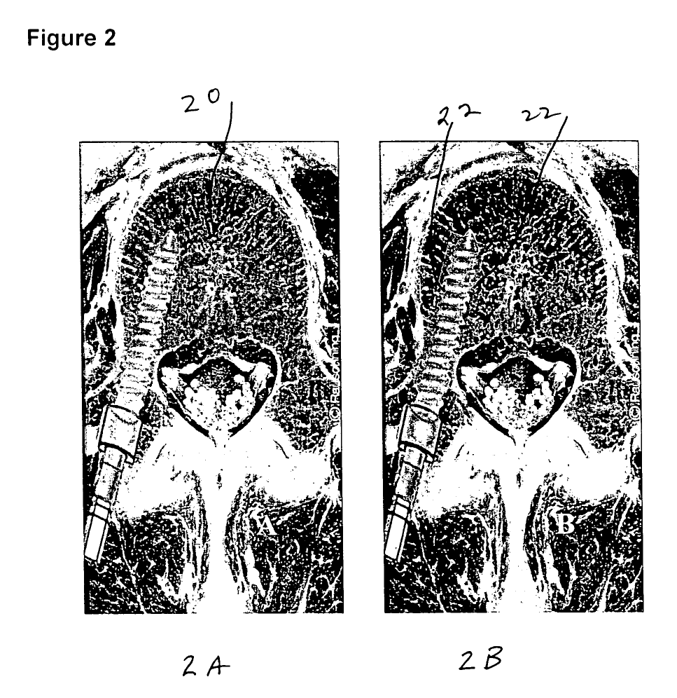

FIG. 2 shows the cross-section of lumbar vertebra with rendition of a pedicle screw. FIG. 2a shows the global bone density 20 which measures all bone in the vertebral body, while FIG. 2b highlights only that portion of the bone surrounding the screw 22, which is important to screw pullout.

As shown in FIG. 2b, the bone surrounding the screw is most important for determining screw pullout. The generation of Hounsfield units will be employed to calculate bone density in potential paths through which the screw will pass. This is a more limited measurement in that a smaller area is being analyzed and such area is being analyzed on a pixel or voxel real-time basis. Such generation of Hounsfield units which are converted into actual bone density is conducted real-time while the surgeon is operating on the patient.

Image-guided fine surgery, as described above, has been directed to calculating bone density for screw path optimization. In addition, other real time image-guidance information is also part of the present invention. Such other information can relate to the preferred instrument path for a cannula or other instrument; can visualize small fractures; all of which are relevant to choosing the most effective instrument path.

For instance, other information may also be incorporated into intraoperative image-guidance. Another example is SPECT (single photon emission computed tomography), a radionuclide study indicating metabolically active bone, such as surrounding a fracture site. This may allow the direction of minimally-invasive vertebroplasty (or other bone injections) directly into a fracture site.

This invention comprises a standard, image-guidance workstation and hardware. Software will input QCT data for the anatomy of interest. For example, the lumbar spine would be scanned using the QCT technique and the data is uploaded to the workstation. Computation is automated in software to detect the standards on each slice, generate the regression and convert each voxel or pixel from Hounsfield units to bone density. The latter values are then displayed in grayscale for the generation of images for use by the surgeon.

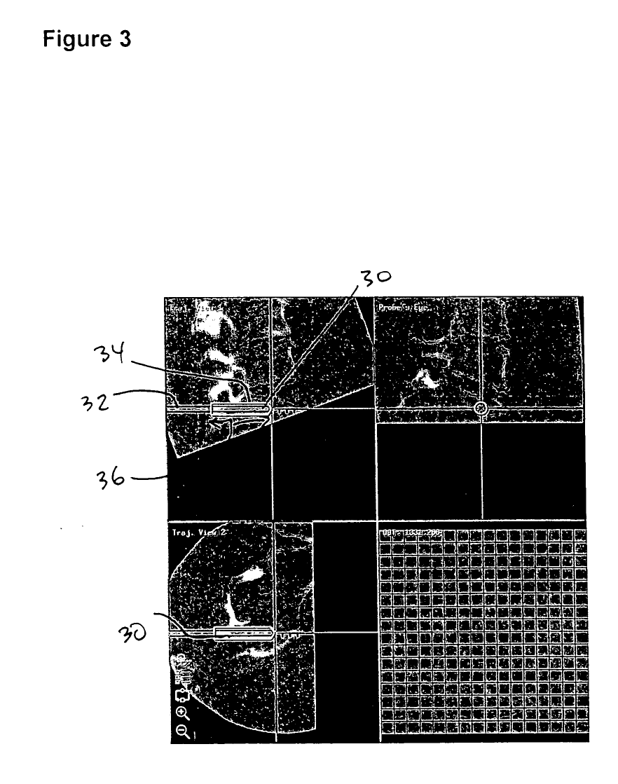

FIG. 3 shows potential screw trajectories with configurable inner and outer diameters, generally describing a hollow cylinder. The lower right quadrant displays average Hounsfield units (HU). Other screw trajectories cause varying average (HU) and, therefore, may be associated with varying pullout strengths. The most effective screw trajectory depending upon bone density and other factors will be chosen and can be calculated.

Further referring to FIG. 3, for planning purposes, screw trajectories can be defined with both an inner 32 and outer 34 diameter, as well as a length 36. These values will define a hollow cylinder. The dimensions of the cylinder reflect that portion of the patient's bone that the surgeon feels is important in screw pullout. The inner diameter will usually reflect the minor diameter of the screw. The outer diameter can be varied, depending on scientific studies as to the relevant participating bone in anchoring the screw. The software displays, real-time, the average bone density and aggregate bone density for this cylinder. These values are used to select a screw path optimized for pullout strength.

An example of vertebral pedicle screws 30 is provided in FIG. 3. Several anatomically valid screw paths are possible for the S1 screws 30 in FIG. 3. However, one has a higher average bone density in the bone surrounding bone and would have a higher pullout strength further referencing the area surrounding the screw in FIG. 2b. Thus, this invention determines these values and selects such a path optimization.

It should be understood that the preferred embodiment was described to provide the best illustration of the principles of the invention and its practical application to thereby enable one of ordinary skill in the art to utilize the invention in various embodiments and with various modifications as are suited to the particular use contemplated. All such modifications and variations are within the scope of the invention as determined by the appended claims when interpreted in accordance with the breadth to which they are fairly legally and equitably entitled.

1. An intra-operative image guidance system for real time visualization of bone properties comprising:
an image display to display a surgical region of interest,
means to conduct a surgical procedure localized to a specific path displaying selected bone properties, and
means to select from alternative paths for an intraoperative surgical procedure the most effective path for such surgical procedure.

2. An intra-operative image guidance system according to claim 1, wherein said system conducts real-time visualization of non-anatomic bone properties.

3. An intra-operative image guidance system according to claim 2, wherein said non-anatomic bone property comprises bone density.

4. An intra-operative image guidance system according to claim 3, wherein bone density is determined on specific pixel/voxel information pertaining to potential paths for screws.

5. An intra-operative image guidance system according to claim 3, wherein said system calculates and displays anatomically correct bone densities.

6. An intra-operative image guidance system according to claim 5, wherein said anatomically correct bone densities are determined on a real-time basis.

7. An intra-operative image guidance system according to claim 2, wherein said surgical procedure includes the insertion of a surgical screw within the skeletal system and further comprises generating screw path densities from relevant bone surrounding the potential screw path, whereby optimization of screw path for pullout strength is determined.

8. An intra-operative image guidance system according to claim 7, wherein said system further comprises means to determine screw trajectory, screw length and/or inner and outer dimensions thereof.

9. An intra-operative image guidance system according to claim 1, wherein said surgical procedure comprises insertion of a surgical instrument, further comprising performing SPECT indicating metabolically active bone.

10. An intra-operative image guidance system according to claim 9, wherein said bone properties are non-anatomic.
